(19) United States
(12) Patent Application Publication
(10) Pub. No.: US 2023/0420073 A1
(43) Pub. Date: Dec. 28, 2023

(54) MACHINE LEARNING MODELS FOR DETERMINING PATHOGENIC GENETIC VARIANTS

(71) Applicant: Gene Friend Way, Inc., San Francisco, CA (US)

(72) Inventors: Duyen Thanh Bui, Hanoi (VN); Tuan Anh Cao, Hanoi (VN); Giang Vu Thanh Pham, Nha Trang (VN)

(73) Assignee: Gene Friend Way, Inc., San Francisco, CA (US)

(21) Appl. No.: 17/849,653

(22) Filed: Jun. 26, 2022

(51) Int. Cl. G16B 20/20; G06N 5/02; G16B 40/20

(52) U.S. Cl. CPC G16B 20/20; G06N 5/022; G16B 40/20

(57) ABSTRACT

A system for determining pathogenic genetic variants is described. The system includes: a knowledge extraction engine configured to: invoke a data crawler to identify research publications related to human genomes of a particular population, for each research publication, analyze content of the research publication to determine whether the respective genetic variant is classified as pathogenic, and in response to determining that the respective genetic variant is classified as pathogenic, add the respective genetic variant to a current set of pathogenic genetic variants; a machine learning model configured to: for each pathogenic genetic variant in the current set, assign a respective importance score to the pathogenic genetic variant based on characteristics of research publications from which the pathogenic genetic variant is determined, and rank the pathogenic genetic variants in the current set according to the respective importance scores; and a variant database configured to store the ranked pathogenic genetic variants.

### BACKGROUND

This disclosure relates to machine learning models for determining pathogenic genetic variants.

A machine learning model is a computer program that can recognize patterns or make decisions from previously unseen data. To perform such tasks, machine learning models are trained with a large training dataset. Once trained, a machine learning model can receive an input and generates an output such as a predicted output based on the received input. Parametric machine learning models are models that generate the output based on the received input and on values of the parameters of the model.

A gene is the basic physical and functional unit of heredity, which refers to the passing on of physical or mental characteristics genetically from one generation to another. Genes are composed of deoxyribonucleic acid (DNA), which is a genetic code that allows a living being to produce proteins. There are approximately 20,000-25,000 genes in human cells. The information in these genes is inherited from each parent and each human has two copies of each gene: one from a father and one from a mother.

A genetic variant is a permanent change in the DNA sequence that makes up a gene. Variants can affect undergo mutations, which are changes in the genetic code that may affect the function of a specific gene. There are two major types of mutations: (i) hereditary (or germline) mutations which are inherited mutations presenting in reproductive cells (eggs or sperms), which are found in the DNA of every cell in the body of an offspring, and (ii) somatic mutations, which occur after conception as a result of environmental factors such as sunlight or due to errors in a DNA replication.

### SUMMARY

This specification describes a machine learning system implemented as computer programs on one or more computers in one or more locations that includes a knowledge extraction engine, a machine learning model and a variant database and that is configured to determine pathogenic genetic variants specific to a particular population.

The subject matter described in this specification can be implemented in particular embodiments so as to realize one or more of the following advantages. The techniques described herein provides a machine learning system that can automatically detect pathogenic genetic variants in a low-cost and efficient manner while still achieving results that have high accuracy. In particular, by including a knowledge extraction engine, a machine learning model, and a variant database, the machine learning system described herein can analyze and identify genetic variants of interest (e.g., those that are associated with harmful hereditary mutations) more accurately than genotyping and more cost-effective and more computationally efficient than sequencing. In addition, the described machine learning system can detect pathogenic genetic variants specific to a particular population (e.g., Asian population, African population, Hispanic population or any other specific population). This is a significant technical improvement to state of the art systems because in current clinical settings, risk prediction algorithms employed by existing systems detect disorders mainly based on a combination of lifestyle, family history, environmental, age, gender and physiological factors. However, these types of algorithms fail to account for important genetic factors and may underestimate or overestimate the risk of disease for certain subgroups (e.g., Asian, African, Caucasian, or Hispanic population).

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example machine learning system for determining pathogenic genetic variants.

FIG. 2 is a flow diagram of an example process for determining pathogenic genetic variants.

Like reference numbers and designations in the various drawings indicate like elements.

### DETAILED DESCRIPTION

This specification describes a machine learning system implemented as computer programs on one or more computers in one or more locations that is configured to determine pathogenic genetic variants specific to a particular population.

Generally, a genetic variant is a permanent change in the DNA sequence that makes up a gene. Variants can affect undergo mutations, which are changes in the genetic code that may affect the function of a specific gene. There are two major types of mutations: (i) hereditary (or germline) mutations which are inherited mutations presenting in reproductive cells (e.g., eggs or sperms), which are found in the DNA of every cell in the body of an offspring, and (ii) somatic mutations, which occur after conception as a result of one or more environmental factors (e.g., sunlight) or due to errors in a DNA replication. Unlike somatic mutations which are not passed on to offspring, hereditary mutations may be inherited by an offspring from its parents. Thus, finding harmful hereditary mutations is imperative for clinical medicine as changes in the genetic code can confer protection from, or increased risk of, disease as well as possible drug resistance and changes in biomarkers relevant to diagnostics.

Previous methods have used either genotyping or sequencing technique in gene decoding in order to identify pathogenic genetic variants that may result in harmful hereditary mutations. Genotyping methods identify specific genetic variants within an individual by looking at targeted, known areas of a person's genome in order to identify those variants. Sequencing methods, on the other hand, look at larger sections of, or the entire, genome in order to identify known genetic variants as well as new variants.

However, both genotyping and sequencing methods have many technical drawbacks. While sequencing offers good discovery power and sensitivity for rare or new variants and is useful for cases where many target regions need to be analyzed, sequencing is highly time-consuming and expensive in both computational resources and monetary costs. In some cases, it may take weeks and cost thousands to tens of thousands of dollars to sequence one genome. The sequencing process also consumes a large amount of computational resources and data storage space. In addition, most of the data derived from sequencing is hard to perceive and therefore is difficult to use in practice. This results in much of the cost being wasted on sequencing regions of the genome that are of little use.

Although genotyping is cheaper than sequencing, its breadth and depth of coverage and accuracy are lower than sequencing. In particular, genotyping requires prior knowledge of the variants of interest and therefore can miss other important variants that are not tested for or have not been described in literature as related to a specific disorder. Further, genotyping may not be able to capture specific types of mutations such as copy number variants. This means, in some cases, genotyping may not provide enough accurate information for the identification of a mutation that may explain a person's disorder or risks.

To overcome the technical drawbacks of genotyping and sequencing methods, the subject matter described in this application provides a machine learning system that can automatically detect pathogenic genetic variants in a low-cost and efficient manner while still achieving results that have high accuracy. In particular, by including a knowledge extraction engine, a machine learning model, and a variant database, the machine learning system described herein can analyze and identify genetic variants of interest (e.g., those that are associated with harmful hereditary mutations) more accurately than genotyping and more cost-effective and more computationally efficient than sequencing.

In addition, the described machine learning system can accurately detect pathogenic genetic variants specific to a particular population (e.g., Asian population, African population, Hispanic population, Caucasian population or any other specific population) by using a knowledge extraction engine that automatically finds and analyzes a large number (e.g., hundreds of thousands or millions) of publications (e.g., research papers, articles, news, reports, etc.) related to the target population. This technique provides a significant technical improvement over state-of-the-art systems because in current clinical settings, risk prediction algorithms employed by existing systems detect disorders mainly based on a combination of lifestyle, family history, environmental, age, gender and physiological factors. These types of algorithms fail to account for important genetic factors and may underestimate or overestimate the risk of disease for certain subgroups (e.g., Asian, African, Caucasian, or Hispanic population).

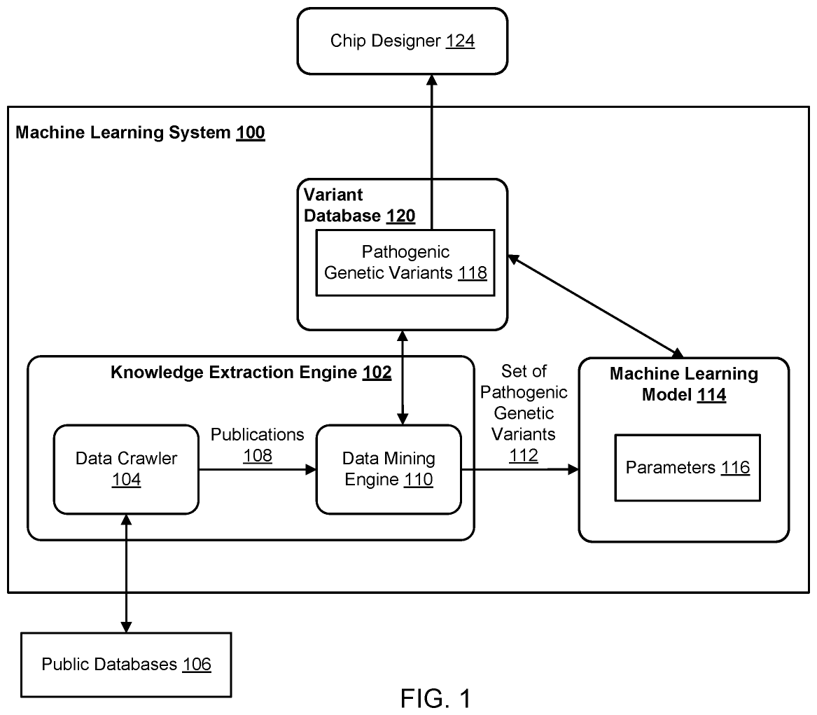

FIG. 1 shows an example machine learning system 100. The machine learning system 100 is an example of a system implemented as computer programs on one or more computers in one or more locations, in which the systems, components, and techniques described below can be implemented.

The machine learning system 100 is configured to determine pathogenic genetic variants specific to a particular population. In some implementations, the particular population is Asian population. In some other implementations, the particular population is another population (e.g., African population, Hispanic, or Caucasian population). The machine learning system 100 includes a knowledge extraction engine 102, a machine learning model 114, and a variant database 120.

Each of the knowledge extraction engine 102 and the machine learning model 114 is implemented as one or more software modules or components, installed on one or more computers in one or more locations. In some cases, one or more computers will be dedicated to a particular engine; in other cases, multiple engines can be installed and running on the same computer or computers. The variant database 120 can be a local database that resides in one or more local systems (e.g., computer systems of an organization), or a distributed database of a cloud computing system.

In some implementations, the machine learning model 114 is a neural network that includes one or more neural network layers, which are composed by interconnected artificial neurons. The one or more neural network layers are nonlinear units that predict an output for a received input. The one or more neural network layers may include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

The knowledge extraction engine 102 is configured to invoke, using an application program interface (API), a data crawler 104 to identify publications related to human genomes of the particular population. The API may be executed by a plurality of servers in a distributed computing system. The API is a set of computer protocols that enables the knowledge extraction engine 102 to communicate and exchange data with the data crawler 104. Each of the plurality of publications refers to a respective genetic variant in a set of genetic variants. The set of genetic variants includes genetic variants of interest, for example, those that are related to the particular population. The publications may include research publications such as scientific papers, theses, articles, and reports. The publications may also include other types of publications such as news and social media posts.

The data crawler 104 is implemented as one or more software modules or components, installed on one or more computers in one or more locations. In some cases, one or more computers will be dedicated to a particular engine; in other cases, multiple engines can be installed and running on the same computer or computers. The data crawler 104 is configured to visit publications (e.g., those that are published on the Internet) that are linked to each other and index new information. More specifically, the data crawler 104 visits relevant publications that are cited by the publications that it already visited. The data crawler 104 determines whether each of the relevant publications mentions a new genetic variant that is not mentioned by the previous publications found by the data crawler 104. If a new genetic variant is mentioned, the data crawler 104 creates a new index for the new genetic variant and includes the publication that mentions the new genetic variant in the list of publication 108 to be sent to a data mining engine 110 for further analysis. In some implementation, the data crawler 104 determines whether each of the relevant publications mentions new information about an existing genetic variant previously found by the data crawler 104, and if so, the data crawler 104 updates an index corresponding to the existing genetic variant and includes the publication in the list of publications 108 to be sent to a data mining engine 110 for further analysis.

The data mining engine 110 is implemented as one or more software modules or components, installed on one or more computers in one or more locations. In some cases, one or more computers will be dedicated to a particular engine; in other cases, multiple engines can be installed and running on the same computer or computers. For each of the publications 108, the data mining engine 110 is configured to analyze content of the research publication to determine whether the respective genetic variant is classified as a pathogenic genetic variant according to the research publication.

In some implementations, the content of each research publication includes text. In these implementations, for each research publication, to determine whether the respective genetic variant is classified as the pathogenic genetic variant, the data mining engine 110 extracts from the text of the research publication a conclusion with respect to the respective genetic variant by using a text mining algorithm. The data mining engine 110 then determines whether the conclusion classifies the respective genetic variant as the pathogenic genetic variant.

In some other implementations, the content of each research publication includes one or more images. The one or more images include additional text. In these implementations, the data mining engine 110 is configured to extract the additional text from the one or more images using optical character recognition and to determine whether the additional text classifies the respective genetic variant as the pathogenic genetic variant.

In response to determining that the respective genetic variant is classified as the pathogenic genetic variant, the knowledge extraction engine 102 is configured to (i) add the respective genetic variant to a current set of pathogenic genetic variants 112, (ii) determine, from the content of the research publication, a phenotype that the respective genetic variant is linked to, and (ii) determine one or more characteristics of the research publication. A phenotype is a set of observable characteristics of an individual resulting from the interaction of its genotype 1o with the environment. The one or more characteristics of the research publication include one or more of: (a) a size of a research study associated with the research publication; (b) a number of times that the research publication has been cited by other publications or other data sources; (c) a p-value or a z-score that represents quality of test results derived by the research study; or (d) a confidence interval of research findings described in the research publication.

The machine learning model 114 is configured to, for each pathogenic genetic variant in the current set of pathogenic genetic variants 112, assign a respective importance score to the pathogenic genetic variant based on characteristics of research publications from which the pathogenic genetic variant is determined. The respective importance score represents (i) a level of importance of the pathogenic genetic variant to the particular population and (ii) a level of contribution of the pathogenic genetic variant to the phenotype that the pathogenic genetic variant is linked to.

In particular, in some implementations, to determine a respective importance score for each pathogenic genetic variant, the machine learning model 114 extracts, from the content of the research publication, data specifying an explanation of a biological reasoning behind the pathogenic genetic variant. The machine learning model 114 assigns a respective importance score to the pathogenic genetic variant based on characteristics of research publications from which the pathogenic genetic variant is determined and based on the explanation of the biological reasoning behind the pathogenic genetic variant.

In some other implementations, for each pathogenic genetic variant in the current set of pathogenic genetic variants 112, the machine learning model 114 is configured to assign the respective importance score to the pathogenic genetic variant using a decision tree induction technique in accordance with one or more parameters 116 of the machine learning model. The one or more parameters 116 include one or more of (i) a first parameter representing a clinical effect, (ii) a second parameter representing a number of validations of a research study, (iii) a third parameter representing a size of the research study, (iv) a fourth parameter representing at least one of a p-value, a z-score, or a confidence interval, (v) a fifth parameter representing a variant prevalence, or (vi) a sixth parameter representing metadata of the research study.

The machine learning model 114 then combines the set of pathogenic genetic variants 112 with the current set of pathogenic genetic variants 118 that is stored in the variant database 120 and ranks all variants according to the respective importance scores of all variants. The machine learning model 114 then updates the variant database 120 with the newly ranked pathogenic genetic variants. This newly ranked pathogenic genetic variants becomes the current set of pathogenic genetic variants 118.

The variant database 120 may send the current set of ranked pathogenic genetic variants 118 to a chip designer 124 that uses the ranked pathogenic genetic variants 118 to construct a chip configured to decode human genomes of individuals in the particular population.

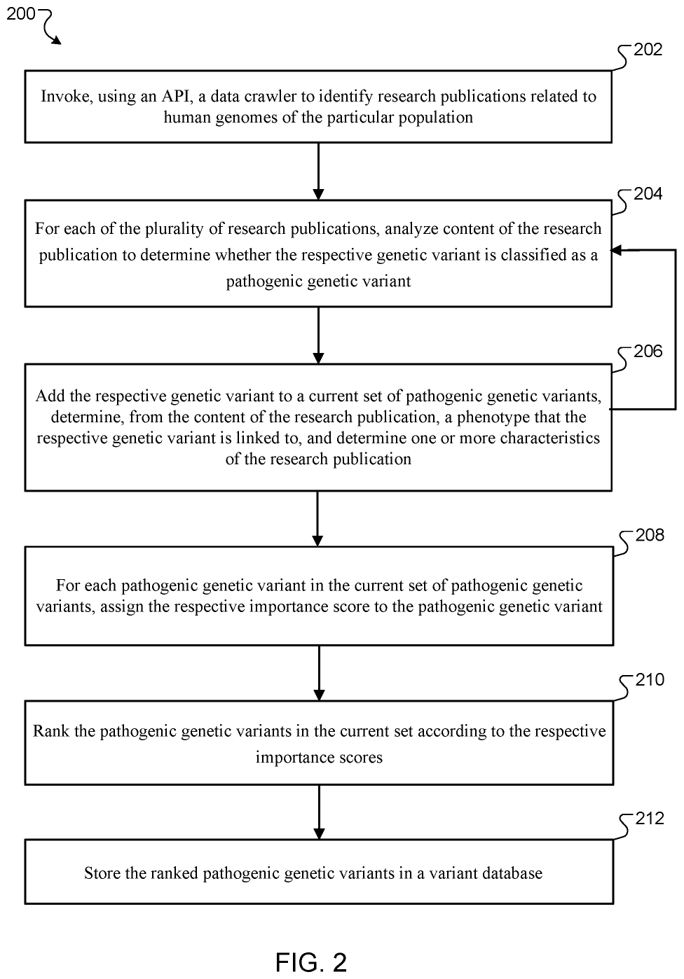

FIG. 2 is a flow diagram of an example process 200 for determining pathogenic genetic variants specific to a particular population. In some implementations, the particular population is Asian population. In some other implementations, the particular population is another population (e.g., African population, Hispanic, or Caucasian population).

For convenience, the process 200 will be described as being performed by a system of one or more computers located in one or more locations. For example, a machine learning system, e.g., the machine learning system 100 of FIG. 1, appropriately programmed in accordance with this specification, can perform the process 200.

The system invokes, using an application program interface (API), a data crawler to identify research publications related to human genomes of the particular population (step 202). The API may be executed by a plurality of servers in a distributed computing system. Each of the plurality of publications refers to a respective genetic variant in a set of genetic variants. The set of genetic variants includes genetic variants of interest, i.e., those that related to the particular population. The publications may include research publications such as scientific papers, theses, articles, and reports. The publications may also include other types of publications such as news and social media posts.

The data crawler is configured to visit publications (e.g., those that are published on the Internet) that are linked to each other and index new information. More specifically, the data crawler visits relevant publications that are cited by the publications that it already visited. The data crawler determines whether each of the relevant publications mentions a new genetic variant that is not mentioned by the previous publications found by the data crawler. If a new genetic variant is mentioned, the data crawler 104 creates a new index for the new genetic variant and includes the publication that mentions the new genetic variant in the list of publication to be sent to a data mining engine for further analysis. In some implementation, the data crawler determines whether each of the relevant publications mentions new information about an existing genetic variant previously found by the data crawler, and if so, the data crawler updates an index corresponding to the existing genetic variant and includes the publication in the list of publications to be sent to a data mining engine for further analysis.

For each of the plurality of research publications, the system performs steps 204-206 as follows.

The system analyzes content of the research publication to determine whether the respective genetic variant is classified as a pathogenic genetic variant according to the research publication (step 204).

In some implementations, the content of each research publication includes text. In these implementations, for each research publication, to determine whether the respective genetic variant is classified as the pathogenic genetic variant, the system extracts from the text of the research publication a conclusion with respect to the respective genetic variant by using a text mining algorithm. The system then determines whether the conclusion classifies the respective genetic variant as the pathogenic genetic variant.

In some other implementations, the content of each research publication includes one or more images. The one or more images include additional text. In these implementations, the system extracts the additional text from the one or more images using optical character recognition and to determine whether the additional text classifies the respective genetic variant as the pathogenic genetic variant.

In response to determining that the respective genetic variant is classified as the pathogenic genetic variant, the system (i) adds the respective genetic variant to a current set of pathogenic genetic variants, (ii) determines, from the content of the research publication, a phenotype that the respective genetic variant is linked to, and (ii) determines one or more characteristics of the research publication (step 206). A phenotype is a set of observable characteristics of an individual resulting from the interaction of its genotype with the environment. The one or more characteristics of the research publication include one or more of: (a) a size of a research study associated with the research publication; (b) a number of times that the research publication has been cited by other publications or other data sources; (c) a p-value or a z-score that represents quality of test results derived by the research study; or (d) a confidence interval of research findings described in the research publication.

For each pathogenic genetic variant in the current set of pathogenic genetic variants, the system assigns a respective importance score to the pathogenic genetic variant based on characteristics of research publications from which the pathogenic genetic variant is determined (step 208). The respective importance score represents (i) a level of importance of the pathogenic genetic variant to the particular population and (ii) a level of contribution of the pathogenic genetic variant to the phenotype that the pathogenic genetic variant is linked to.

In particular, in some implementations, to determine a respective importance score for each pathogenic genetic variant, the system extracts, from the content of the research publication, data specifying an explanation of a biological reasoning behind the pathogenic genetic variant.

The system assigns, using a machine learning model, a respective importance score to the pathogenic genetic variant based on characteristics of research publications from which the pathogenic genetic variant is determined and based on the explanation of the biological reasoning behind the pathogenic genetic variant.

In some other implementations, for each pathogenic genetic variant in the current set of pathogenic genetic variants, the system is configured to assign the respective importance score to the pathogenic genetic variant using a decision tree induction technique in accordance with one or more parameters of the machine learning model. The one or more parameters of the machine learning model include one or more of (i) a first parameter representing a clinical effect, (ii) a second parameter representing a number of validations of a research study, (iii) a third parameter representing a size of the research study, (iv) a fourth parameter representing at least one of a p-value, a z-score, or a confidence interval, (v) a fifth parameter representing a variant prevalence, or (vi) a sixth parameter representing metadata of the research study.

The system ranks the pathogenic genetic variants in the current set according to the respective importance scores (step 210).

The system stores the ranked pathogenic genetic variants in a variant database (step 212).

Optionally, when the variant data already stores a set of pathogenic genetic variants, the system combines the set of pathogenic genetic variants that it has ranked with the set of pathogenic genetic variants currently stored in the variant database and ranks all variants according to the respective importance scores of all variants. The system then updates the variant database with the newly ranked pathogenic genetic variants. This newly ranked pathogenic genetic variants becomes the current set of pathogenic genetic variants of the variant database.

In some implementations, the system sends the current set of ranked pathogenic genetic variants stored in the variant database to a chip designer that uses the ranked pathogenic genetic variants to construct a chip configured to decode human genomes of individuals in the particular population.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them.

Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions.

Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated 1o in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A system for determining pathogenic genetic variants specific to a particular population, the system comprising:
a knowledge extraction engine configured to:
invoke, using an application program interface (API), a data crawler to identify research publications related to human genomes of the particular population, wherein each of the plurality of research publications refers to a respective genetic variant of a plurality of genetic variants,
for each of the plurality of research publications,
analyze content of the research publication to determine whether the respective genetic variant is classified as a pathogenic genetic variant according to the research publication, and
in response to determining that the respective genetic variant is classified as the pathogenic genetic variant, (i) add the respective genetic variant to a current set of pathogenic genetic variants, (ii) determine, from the content of the research publication, a phenotype that the respective genetic variant is linked to, and (ii) determine one or more characteristics of the research publication;
a machine learning model configured to:
for each pathogenic genetic variant in the current set of pathogenic genetic variants, assign a respective importance score to the pathogenic genetic variant based on characteristics of research publications from which the pathogenic genetic variant is determined, wherein the respective importance score represents (i) a level of importance of the pathogenic genetic variant to the particular population and (ii) a level of contribution of the pathogenic genetic variant to the phenotype that the pathogenic genetic variant is linked to, and
rank the pathogenic genetic variants in the current set according to the respective importance scores; and
a variant database configured to store the ranked pathogenic genetic variants.

2. The system of claim 1, wherein the particular population is Asian population.

3. The system of claim 1, wherein the content of each research publication includes text, and
wherein for each research publication, analyzing the content of the research publication to determine whether the respective genetic variant is classified as the pathogenic genetic variant comprises:
extracting, from the text of the research publication, a conclusion with respect to the respective genetic variant by using a text mining algorithm; and
determining whether the conclusion classifies the respective genetic variant as the pathogenic genetic variant.

4. The system of claim 1, wherein the content of each research publication includes one or more images, the one or more images including second text, and
wherein for each research publication, analyzing the content of the research publication to determine whether the respective genetic variant is classified as the pathogenic genetic variant comprises:
extracting the second text from the one or more images using optical character recognition; and
determining whether the second text classifies the respective genetic variant as the pathogenic genetic variant.

5. The system of claim 1, wherein the one or more characteristics of the research publication include one or more of:
(a) a size of a research study associated with the research publication;
(b) a number of times that the research publication has been cited by other publications or other data sources;
(c) a p-value that represents quality of test results derived by the research study; or
(d) a confidence interval of research findings described in the research publication;

6. The system of claim 1, wherein the knowledge extraction engine is further configured to:
for each of the plurality of research publications, in response to determining that the respective genetic variant is classified as the pathogenic genetic variant:
extracting, from the content of the research publication, an explanation of a biological reasoning behind the pathogenic genetic variant; and
wherein the machine learning model is configured to:
for each pathogenic genetic variant in the current set of pathogenic genetic variants, assign a respective importance score to the pathogenic genetic variant based on characteristics of research publications from which the pathogenic genetic variant is determined and based on the explanation of the biological reasoning behind the pathogenic genetic variant.

7. The system of claim 1, wherein the machine learning model has one or more parameters, wherein the one or more parameters include one or more of (i) a first parameter representing a clinical effect, (ii) a second parameter representing a number of validations of a research study, (iii) a third parameter representing a size of the research study, (iv) a fourth parameter representing at least one of a p-value, a z-score, or a confidence interval, (v) a fifth parameter representing a variant prevalence, or (vi) a sixth parameter representing metadata of the research study.

8. The system of claim 7, wherein, for each pathogenic genetic variant in the current set of pathogenic genetic variants, the machine learning model is configured to assign the respective importance score to the pathogenic genetic variant using a decision tree induction technique in accordance with the one or more parameters of the machine learning model.

9. The system of claim 1, wherein the ranked pathogenic genetic variants stored in the variant database is used to construct a chip configured to decode human genomes of individuals in the particular population.

10. A computer-implemented method comprising:
invoking, using an application program interface (API), a data crawler to identify research publications related to human genomes of the particular population, wherein each of the plurality of research publications refers to a respective genetic variant of a plurality of genetic variants;
for each of the plurality of research publications,
analyzing content of the research publication to determine whether the respective genetic variant is classified as a pathogenic genetic variant according to the research publication, and
in response to determining that the respective genetic variant is classified as the pathogenic genetic variant, (i) adding the respective genetic variant to a current set of pathogenic genetic variants, (ii) determining, from the content of the research publication, a phenotype that the respective genetic variant is linked to, and (ii) determining one or more characteristics of the research publication;
for each pathogenic genetic variant in the current set of pathogenic genetic variants, assigning, using a machine learning model, the respective importance score to the pathogenic genetic variant based on characteristics of research publications from which the pathogenic genetic variant is determined, wherein the respective importance score represents (i) a level of importance of the pathogenic genetic variant to the particular population and (ii) a level of contribution of the pathogenic genetic variant to the phenotype that the pathogenic genetic variant is linked to;
ranking, using the machine learning model, the pathogenic genetic variants in the current set according to the respective importance scores; and
storing the ranked pathogenic genetic variants in a variant database.

11. The method of claim 10, wherein the particular population is Asian population.

12. The method of claim 10, wherein the content of each research publication includes text, and
wherein for each research publication, analyzing the content of the research publication to determine whether the respective genetic variant is classified as the pathogenic genetic variant comprises:
extracting, from the text of the research publication, a conclusion with respect to the respective genetic variant by using a text mining algorithm; and
determining whether the conclusion classifies the respective genetic variant as the pathogenic genetic variant.

13. The method of claim 10, wherein the content of each research publication includes one or more images, the one or more images including second text, and
wherein for each research publication, analyzing the content of the research publication to determine whether the respective genetic variant is classified as the pathogenic genetic variant comprises:
extracting the second text from the one or more images using optical character recognition; and
determining whether the second text classifies the respective genetic variant as the pathogenic genetic variant.

14. The method of claim 10, wherein the one or more characteristics of the research publication include one or more of:
(a) a size of a research study associated with the research publication;
(b) a number of times that the research publication has been cited by other publications or other data sources;
(c) a p-value that represents quality of test results derived by the research study; or
(d) a confidence interval of research findings described in the research publication;

15. The method of claim 10, further comprising:
for each of the plurality of research publications, in response to determining that the respective genetic variant is classified as the pathogenic genetic variant:
extracting, from the content of the research publication, an explanation of a biological reasoning behind the pathogenic genetic variant; and
for each pathogenic genetic variant in the current set of pathogenic genetic variants, assigning, using the machine learning model, the respective importance score to the pathogenic genetic variant based on characteristics of research publications from which the pathogenic genetic variant is determined and based on the explanation of the biological reasoning behind the pathogenic genetic variant.

16. The method of claim 10, wherein the machine learning model has one or more parameters, wherein the one or more parameters include one or more of (i) a first parameter representing a clinical effect, (ii) a second parameter representing a number of validations of a research study, (iii) a third parameter representing a size of the research study, (iv) a fourth parameter representing a p-value, a z-score, or a confidence interval, (v) a fifth parameter representing a variant prevalence, or (vi) a sixth parameter representing metadata of the research study.

17. The method of claim 16, wherein, for each pathogenic genetic variant in the current set of pathogenic genetic variants, assigning, using the machine learning model, the respective importance score to the pathogenic genetic variant comprises:
assigning, using the machine learning model, the respective importance score to the pathogenic genetic variant in accordance with the one or more parameters of the machine learning model.

18. The method of claim 10, further comprising:
using the ranked pathogenic genetic variants stored in the variant database to construct a chip configured to decode genomes of individuals in the particular population.

19. One or more non-transitory computer storage media storing instructions that, when executed by one or more computers, cause the one or more computers to perform operations comprising:
invoking, using an application program interface (API), a data crawler to identify research publications related to human genomes of the particular population, wherein each of the plurality of research publications refers to a respective genetic variant of a plurality of genetic variants;
for each of the plurality of research publications,
analyzing content of the research publication to determine whether the respective genetic variant is classified as a pathogenic genetic variant according to the research publication, and
in response to determining that the respective genetic variant is classified as the pathogenic genetic variant, (i) adding the respective genetic variant to a current set of pathogenic genetic variants, (ii) determining, from the content of the research publication, a phenotype that the respective genetic variant is linked to, and (ii) determining one or more characteristics of the research publication;
for each pathogenic genetic variant in the current set of pathogenic genetic variants, assigning, using a machine learning model, the respective importance score to the pathogenic genetic variant based on characteristics of research publications from which the pathogenic genetic variant is determined, wherein the respective importance score represents (i) a level of importance of the pathogenic genetic variant to the particular population and (ii) a level of contribution of the pathogenic genetic variant to the phenotype that the pathogenic genetic variant is linked to;
ranking, using the machine learning model, the pathogenic genetic variants in the current set according to the respective importance scores; and
storing the ranked pathogenic genetic variants in a variant database.

20. The one or more non-transitory computer storage media of claim 19, wherein the particular population is Asian population.
